(19) United States
(12) Patent Application Publication
(10) Pub. No.: US 2026/0120854 A1
(43) Pub. Date: Apr. 30, 2026

(54) SYSTEMS AND METHODS FOR RADIATION ONCOLOGY WORKFLOW MANAGEMENT

(71) Applicant: The General Hospital Corporation, Boston, MA (US)

(72) Inventor: John Wolfgang, Boston, MA (US)

(21) Appl. No.: 19/118,689

(22) PCT Filed: Oct. 6, 2023
(86) PCT No.: PCT/US2023/076210

§ 371 Date: Apr. 4, 2025

Related U.S. Application Data
(60) Provisional application No. 63/378,666, filed on Oct. 6, 2022.

(51) Int. Cl. G16H 40/20; G06F 3/0482; G06F 3/0484; G16H 30/20

(52) U.S. Cl. CPC G16H 40/20; G06F 3/0484; G16H 30/20; G06F 3/0482

(57) ABSTRACT

A radiation oncology workflow management system is provided. The system may include interactive graphical interface and a particularly programmed processor, thereby to perform operations including displaying a window containing a user interface (UI) environment on a computer screen, parsing an input received from a user via the UI environment, thereby to determine which of a plurality of atomic application functions is indicated by the input, automatically interacting with an atomic application associated with the atomic application function determined to be indicated by the input, and automatically displaying information received from the atomic application via the UI environment.

### CROSS-REFERENCE TO RELATED APPLICATIONS

This application claims priority to U.S. Provisional Application No. 63/378,666, titled WORKFLOW MANAGER, filed on Oct. 6, 2022, the entire contents of which are herein incorporated by reference.

### TECHNICAL FIELD

This disclosure relates to the field of healthcare and patient management, for example, in radiation therapy or oncology. More specifically, this disclosure relates to systems and methods for managing workflow and/or coordinating disparate systems and applications in a healthcare for patient or workflow management, for example, in a radiation therapy or oncology environment.

### BACKGROUND

In comparative radiotherapy environments, several disparate systems are generally used. Each system often uses its own software application which is generally provided by the vendors of the individual systems. The different software applications may be written using different coding languages, may implement different interfaces, and may not be capable of easy interoperability with one another.

This constellation of individual applications each providing individual atomic functions within the radiotherapy environments introduces several inefficiencies. In addition to potential incompatibility between the different systems, the disparate nature of the systems results in an increase in the burden for data entry (e.g., the same data may need to be entered in multiple different applications). The increase in data entry requirements may lead to more opportunities for human error, in addition to resulting in increased use of limited computing resources for both data entry and error checking actions. Often, these inefficiencies result in delays before treatment begins, time burden on medical practitioners, and an overall reduced level of care.

### SUMMARY

The present disclosure provides a Radiation Oncology Whiteboard (WB), which provides systems and methods used in, for example, a radiation oncology medical practice to manage clinical events and their associated data. Functionally, the WB defines a sequence of clinical tasks corresponding to a prescribed treatment goal for an indicated patient. Users from different role groups interact with the tasks at different time points, starting, executing, and completing a task. Data from that task may be referenced in context with the task execution and may be passed on to subsequent tasks in the workflow. Interactions with the WB may occur through, for example, a Secure Sockets Layer (SSL) secured Web Browser interface.

According to one aspect of the present disclosure, a radiation oncology workflow management system is provided. The system comprises an interactive graphical interface configured to affect a workflow operation and workstation behavior for a radiation oncology patient-specific workflow; and a particularly programmed processor including computer program instructions, the instructions, when executed by the processor, cause the system to perform: displaying a window containing a user interface (UI) environment on a computer screen, parsing an input received from a user via the UI environment, thereby to determine which of a plurality of atomic application functions is indicated by the input, automatically interacting with an atomic application associated with the atomic application function determined to be indicated by the input, and automatically displaying information received from the atomic application via the UI environment.

According to another aspect of the present disclosure, a non-transitory computer-readable medium is provided. The non-transitory computer-readable medium stores instructions that, when executed by a processor of a computing machine, cause the computing machine to be particularly programmed to perform operations comprising: displaying a window containing a user interface (UI) environment on a computer screen; parsing an input received from a user via the UI environment, thereby to determine which of a plurality of atomic application functions is indicated by the input; automatically interacting with an atomic application associated with the atomic application function determined to be indicated by the input; and automatically displaying information received from the atomic application via the UI environment.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an example in more detail than is necessary for a fundamental understanding of the teachings of the disclosures. In the drawings:

FIGS. 1A and TB respectively illustrate graphical representations of events and data flow in accordance with various aspects of the present disclosure.

FIGS. 2-23 respectively illustrate examples of a graphical user interface (GUI) in accordance with various aspects of the present disclosure.

FIG. 24 illustrates an example of a radiation oncology workflow management system in accordance with various aspects of the present disclosure.

FIG. 25 illustrates an example of data flow in a system in accordance with various aspects of the present disclosure.

### DETAILED DESCRIPTION

The WB set forth herein implements a service-oriented architecture, in which, rather than a single software application performing many functions, a constellation of many applications each of which perform comparatively simple atomic functions are each connected to an orchestrator application (e.g., a workflow engine) that triggers the actions of the atomic applications to complete, in concert, a desired function. In synchrony with event orchestration, the WB provides data orchestration, where it provides and records data to the atomic actors according to the desired functionality.

Thereby, the systems and methods for radiation oncology workflow management set forth herein provides a single interface for control of the entire radiation therapy (RT) care continuum via a customizable ecosystem with complete interoperability with functional platforms. The single interface ensures consistent data and reduces data entry error (e.g., by avoiding any requirement that multiple users each enter redundant data in separate functional platforms), reduces delays in beginning treatment (e.g., by streamlining patient activities and providing automation), and increases the efficiency of medical practitioners (e.g., by reducing administrative burden on doctors and nurses so that more time may be spent with patients). Thus, the systems and methods of the present disclosure effect improvements in several technical fields, including but not limited to radiation oncology. Moreover, by permitting several functional platforms to interface in a streamlined and automated manner, the systems and methods of the present disclosure improve the processing efficiency of the computerized systems themselves (e.g., by reducing resource usage and/or reducing computing downtime).

In order to support the data and event orchestration, WB implements many open standard interfaces for communication with internal and external clinical applications. Interfaces include Digital Imaging and Communications in Medicine (DICOM) (C-FIND. C-STORE, C-MOVE), Health Level Seven (HL7), Representational State Transfer (REST), and Simple Object Access Protocol (SOAP). Workflow, dataflow and interface behavior is customizable. New workflows, including new tasks along with interface descriptions, are described using an architecture referred to herein as Domain Specific Radiation Oncology Workflow Language (ROWL). New workflows described using ROWL may be interactively added to the WB in order to generate new functionality “on the fly,” without interruption of the system.

The WB may include any one or more of the following subsets of applications: a Workflow Engine (Radiation Oncology Collaboration Platform or ROCP), a Workflow User Interface (UI) (CorTx), a Workflow Analytics Application, an Intake Form, a Planning Directive, a Staff Scheduler, a Machine Log Application, a Morning Quality Assurance (QA) Application, a Proton Radiotherapy Application, an Immobilization Device Tracking Application, and a Waiting Room Resource Description Framework Site Summary (RSS) Feed Application.

The ROCP is a workflow orchestrator that maintains a state of all radiotherapy activities and associated data references. It implements business logic for the execution of workflow rules defined in ROWL for patient-specific workflows.

More generally, the ROCP manages and executes business logic for state management of radiation oncology workflow. It provides interfaces for the communication of workflow state and workflow data references to external systems. The ROCP may support interfaces including but not limited to HL7, PHIR, DICOM (C-Move, C-Store, C-Find, UPS, MPPS, Modality Worklist), SOAP and REST external communication interfaces. Workflow is defined using a modeling language developed for the ROCP, referred to as ROWL. ROWL is expressed in XML and processed by an ROCP model executor to support radiotherapy activities. A UI-based ROWL editor is available for creation of new workflows. Because workflows are modeled and not hard-coded, the system is capable of supporting an infinite variety of possible clinical workflow combinations and can be tailored for a specific clinic's existing procedures.

The ROCP may be comprised of an application layer and a data layer, where workflow is connected to dataflow. As data is generated by clinical events, the data references are recorded by the ROCP and published internally such that other workflow tasks may receive these references. The ROCP tasks subscribe to data flows, such that they listen for data to become available, they can then use these data references in their independent execution. Likewise the tasks publish their data so that other subscribing tasks may receive the data when it becomes available.

The ROCP tasks present external interfaces allowing clinical applications to transmit state and data references. Specific interfaces include DICOM Image modality (MPPS), such that the WB may act as a Radiology Information System and directly communicate imaging instructions to an imaging modality (e.g., CT, MRI, PET scanners, and the like). The WB also supports DICOM UPS interfaces allowing for direct communication to treatment modalities such as a photon linear accelerator or proton accelerator to directly control treatment delivery using a pre-defined treatment plan. As workflow is modeled, the WB supports industry standard IHE-RO TDW, TDW-II, IPDW and DPDW treatment workflow protocols.

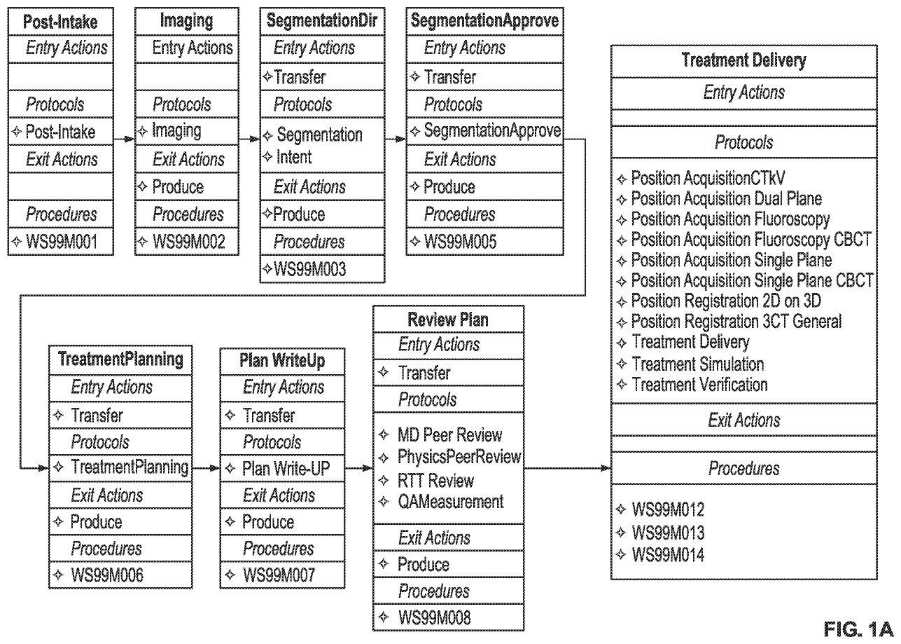

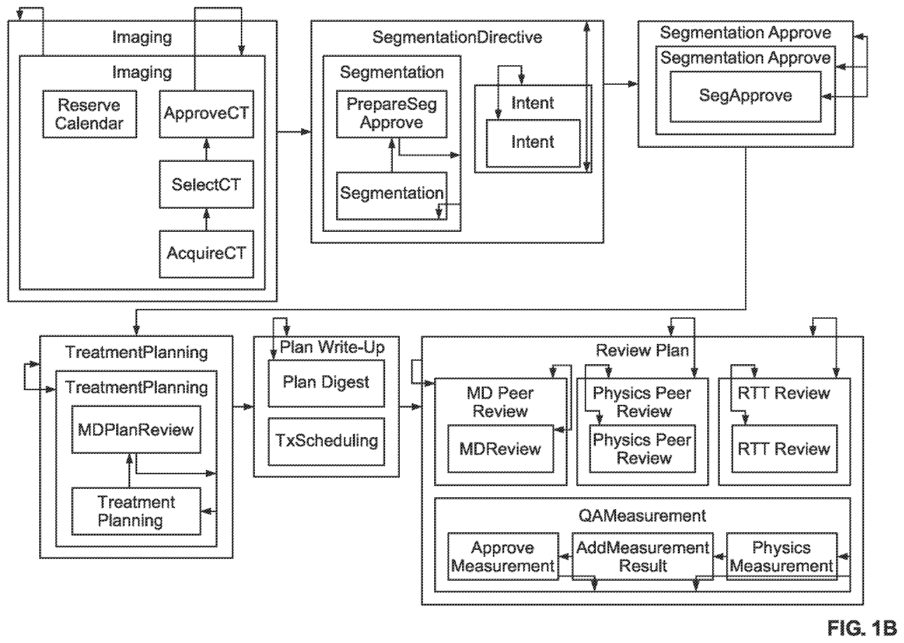

FIGS. 1A and 1B provide example graphical representations of the operation of the ROCP. In particular, FIG. 1A illustrates a graphical representation of ROWL events in an editor, and FIG. 1B illustrates a graphical representation of the dataflow for a publish-subscribe model.

The ROCP may be implemented using Intersystems Iris for Health (InterSystems, Cambridge, MA), which consists of object-oriented non-SQL database with application layer written in Object Script (Intersystems programming language). It may be divided into logical software “layers” defined as: Application, Domain. Interface, and Test. Each horizontal layer may be equivalently subdivided according to one or more data “domains” defined as: Patient, Patient Data Record (PDR), Generic, Storage, Treatment Delivery, Treatment Management, Quality Assurance, Equipment Resource Planning (ERP), and Tracing. The ROCP system may be deployed either locally on a configured host system, or as a cloud-based service using, for example, Kubemetes and Docker containers.

The CorTx UI is a web-based UI that displays the workflow status for all patients as store din the ROCP. It provides controls for filtering/sorting workflow visualizations, interacting with workflows (such as starting, competing, and/or adding data) as defined by the ROWL, an electronic radiation oncology chart view (which, e.g., shows workflows for selected patient and data relationships), and provides tools for user management (such as adding/editing users, specifying user roles, and specifying role permissions).

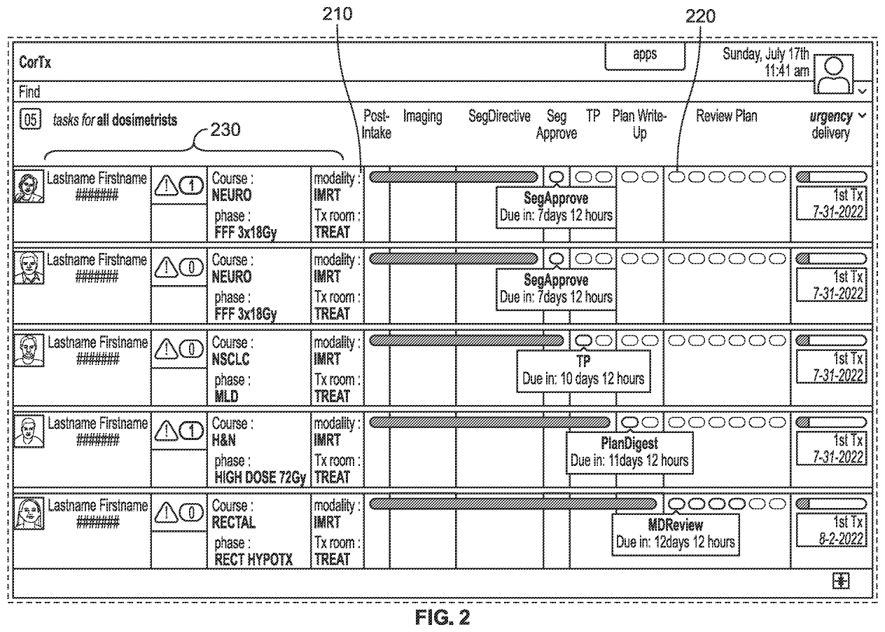

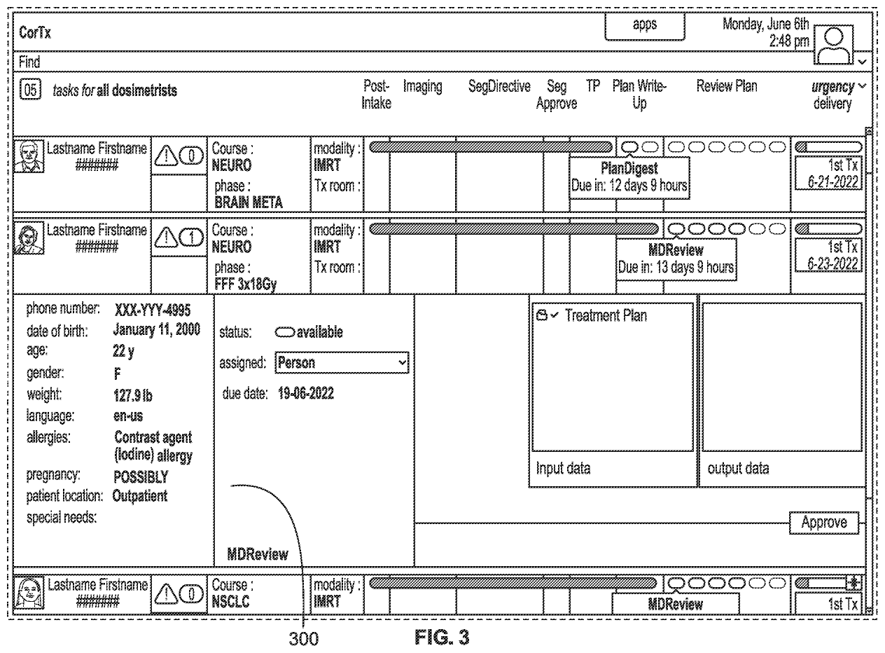

More generally, the CorTx UI provides a graphical, interactive representation of all radiation oncology patient-specific workflows defined for the clinic. FIG. 2 illustrates one example of the CorTx UL. A workflow is displayed in a “racetrack” view where individual tasks appear as “pills” 220 on the horizontal racetrack component 210. In FIG. 2, each horizontal bar is a racetrack including information 230 such as a patient's image, name, medical record number (MRN), medical alert notification, treatment modality, and the work flow track in which each oval or pill corresponds to a clinical task defined in the ROWL definition. A workflow may have different numbers, sequences and organization of pills 220 (tasks) depending on the definition of the workflow (see ROWL above). A pill icon can represent state (e.g., unavailable, scheduled, in progress, done), name of task, and/or relative schedule state (e.g., on-time, due, late). Interaction with a pill 220, such as by clicking on the pill 220, brings up a detail panel where the user can see information regarding the task including due date, assigned user, state, type, required task input data and output data produced through execution of a task. This behavior is illustrated in FIG. 3, in which the selection of a pill 220 opens a task detail panel 300 that provides additional information regarding the assigned task. The workflows visible on the screen may be filtered and sorted by task types, workflow types, users assigned to tasks, user roles assigned to tasks, due date and treatment start date.

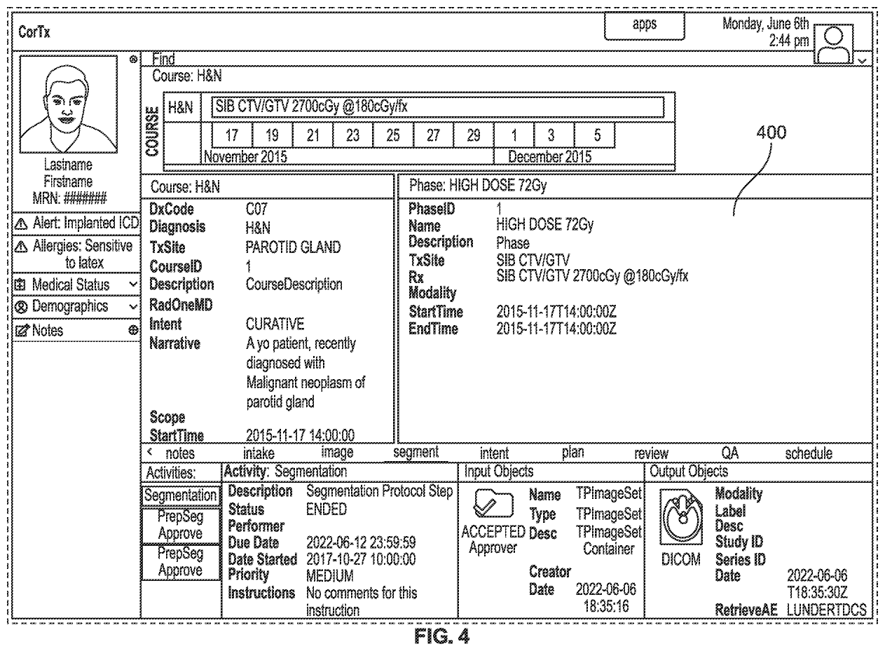

In addition to the racetrack view, the system may present a “chart” view. FIG. 4 illustrates one example of a CorTx chart view. Selection of a patient from the racetrack view may open the chart 400, where information regarding all racetracks is visible. The chart view is interactive, allowing the user to explore task and data relationships for all defined workflows. In the chart view, all workflow data relating to a single selected patient is presented. Data may be organized according to DICOM-RT 2nd Generation schema, where a single workflow relates to a “phase” of a treatment. A collection of phases relates to a “course” of treatment that references a single therapeutic intent (e.g., treatment of Prostate Carcinoma). Data for each phase is presented in the context of the workflow, where a user can select an activity from a racetrack or phase to view the associated data.

The CorTx UI may be implemented as a web application. In one example, the web application may be written using the Angular application framework by (Google, Mountain View, CA). The Angular compiler converts the native TypeScript into JavaScript which is then independently deployable to a modem web server. IIS 7.5+ (Microsoft, Redmond, WA), Apache 2.4+ or Nginx are all supported web servers.

The Workflow Analysis Application is a web-based application that provides statistical views of workflow execution performance. It allows clinical users to sort, filter, and/or review data regarding time-to-complete for workflow events by role, user, medical service, and/or treatment modality.

The Intake Form is a web-based form used by clinicians to enter radiation oncology related data needed for treatment execution. The form may include a general medical questionnaire, a Computed Tomography (CT) Simulation order form, and/or a clinical visit scheduling tool.

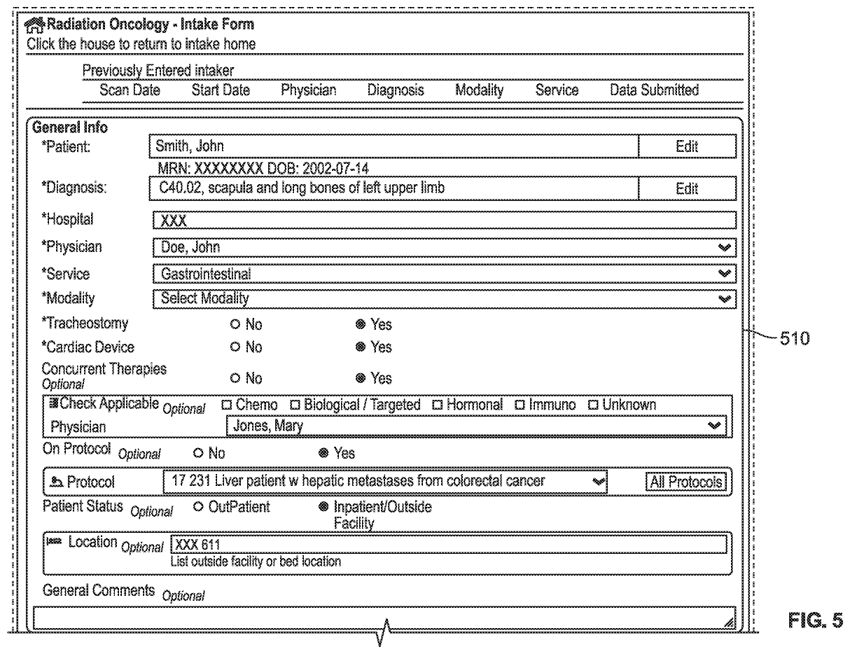

More generally, the Intake Form is an application used to add a patient treatment workflow to the ROCP. The physician or other practitioner fills out the form, answering questions required by other roles in the department in order to start the treatment preparation process. The form is dynamic in nature, such that specific response to questions in the form result in later modifications to the form (new questions, sub-questions, alerts, etc.). The form may be divided into three sections: 1) General Questions, 2) Simulation Order, and 3) Scheduling. FIG. 5 illustrates one example of a patient-specific intake form.

The General Questions section 510 provides fields where patient demographic information and general treatment site information may be entered. In some implementations, the patient name and/or MRN may be retrieved from an Electronic Medical Record (EMR) if available instead of via manual entry. Information including attending physician name, treatment clinic, treatment service, and treatment modality may be entered in this section. The Simulation Order section 520 provides fields where the patient treatment planning simulation, if required for the indicated treatment modality, may be entered. Data from the simulation order may be directly passed to the imaging modality (e.g., CT) using the DICOM Modality Worklist interface. The Scheduling section 530 provides fields where desired dates for planning simulation and treatment start may be entered.

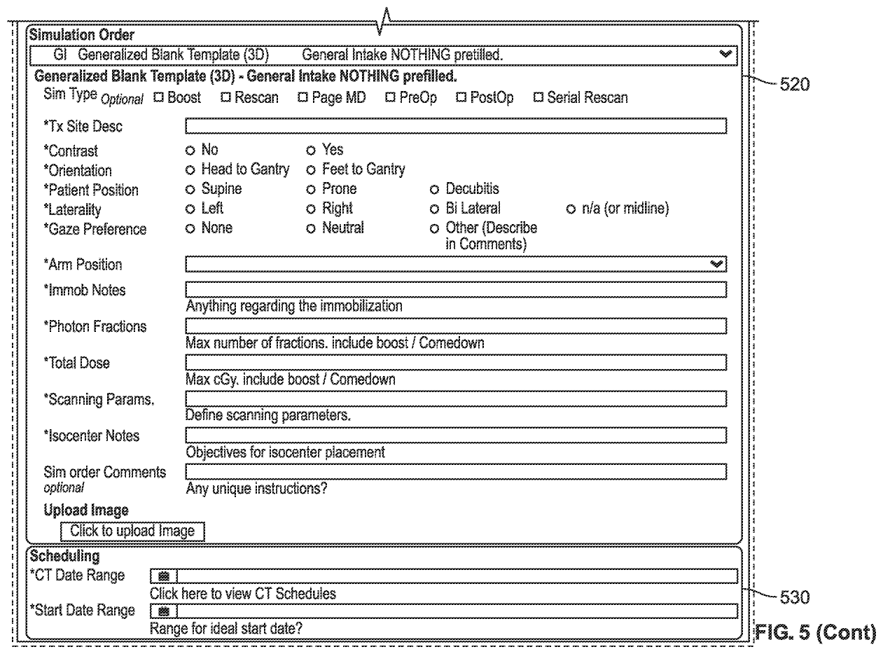

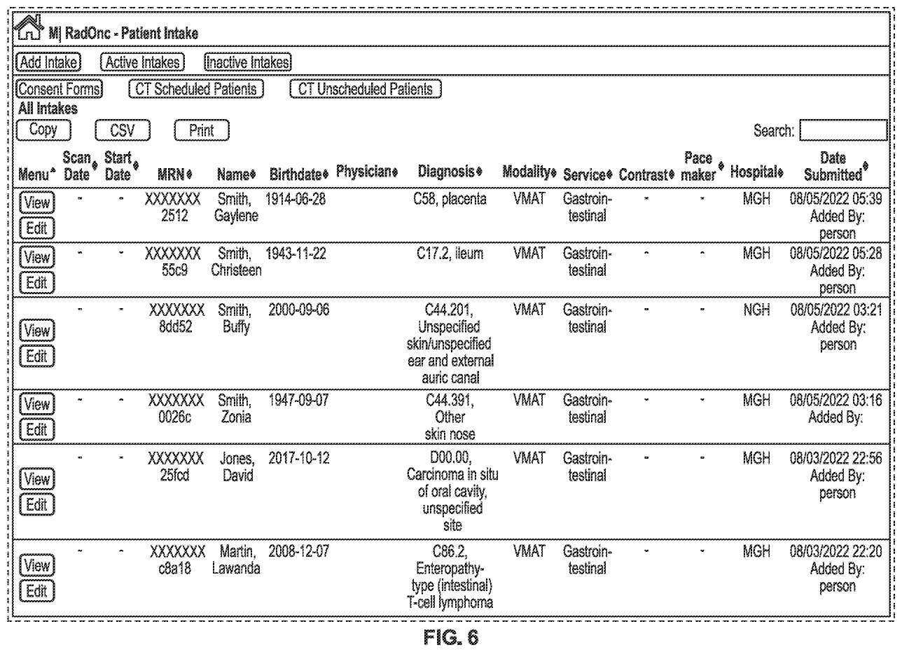

The Intake Form may also provide a list view of all intake forms submitted, as illustrated in FIG. 6. Users may search for and/or view any submitted intake form. Tools for composing new simulation order forms for new treatment modalities may also be provided. Furthermore, the intake form provides functionality for tracking patient referrals. The originating practice that refers a given patient may be added to a list which may be viewed and analyzed for referral patterns. The Intake Form app may be implemented as a combination of PHP server-side scripts and client-side JavaScript, and may be deployed locally on a web server hosted within the hospital network or as a cloud-based application using Docker containers.

The Planning Directive is a web-based form used by clinicians to define radiotherapy planning goals and normal tissue constraints. It may be used by a treatment planner such as a dosimetrist to construct a radiotherapy plan.

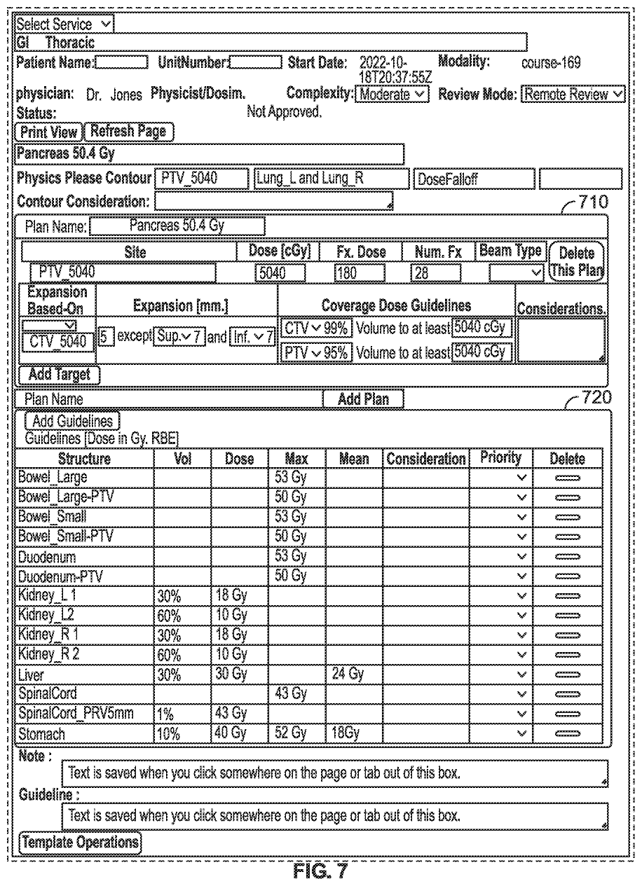

More generally, the Planning Directive application allows a physician to specify for the treatment planner detailed information regarding all dosimetric constraints that should be applied while the planner generates a radiation treatment plan for the patient. One example of a planning guideline form presented via the Planning Directive application is illustrated in FIG. 7. Dose goals to target (tumor) volumes may be specified as well as healthy organ limits. The form may be divided in to “plans” and “guidelines.” The plan section 710 may be further subdivided into targets, such that a single plan may reference multiple treatment targets. The guideline section 720 details all dosimetric constraints by type and numerical limits for healthy organs, to be applied to the sum of all defined plans. The Planning Directive application provides templates (i.e., pre-filled forms), for many common treatment plans reflecting local and national protocols. The application also provides a template editor such that new templates may be generated for new protocols as they are needed. The form highlights any patient specific changes that a physician may introduce from the standard template.

The Planning Directive application may be implemented as a combination of server-side PHP scripts and client-side JavaScript, and may be hosted on a web server within the existing hospital network or as a cloud-based application using Docker containers.

The Staff Scheduler is a web-based UI used to indicate staff task assignments, clinical responsibilities, and availability. It comprises various interactive calendar views allowing staff to self-assign availability and trade clinical assignments as needed. It may include a genetic algorithm-based optimization tool to create a staff schedule based on indicated staff abilities and availabilities.

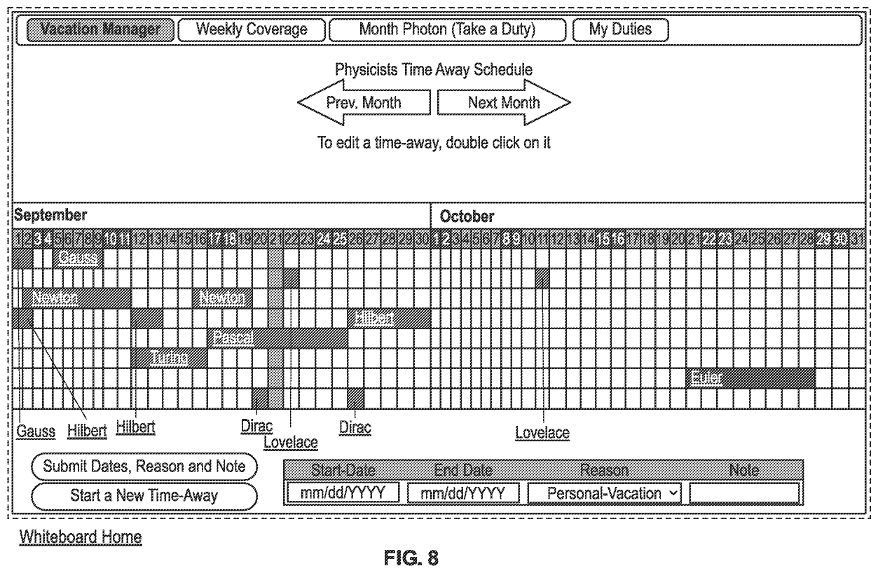

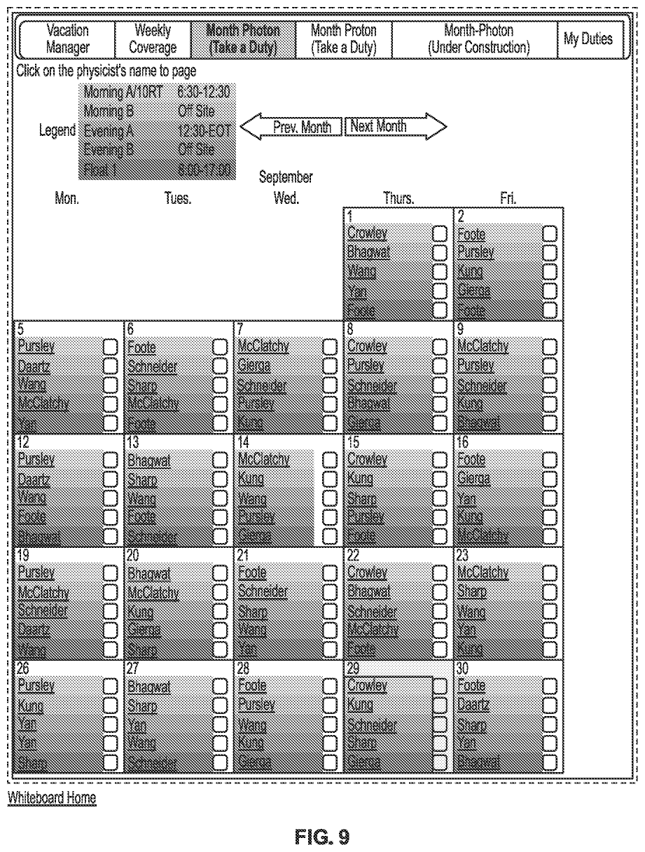

More generally, the Staff Scheduler application provides a means to display and exchange scheduled clinical responsibilities between staff members. Assignments are created using an algorithm-based optimizer that considers the staff capabilities, clinical coverage requirements, and staff availability to generate an initial clinical schedule. The application UI presents the coverage calendar and allows users to exchange responsibilities as needed to address personal requirements. FIGS. 8 and 9 illustrate examples of the application UI. FIG. 8 shows a vacation assignment display, in which horizontal rows reflect staff member availability according to calendar date. FIG. 9 shows a clinical duty assignment display, in which users may trade clinical assignments by “claiming” a shift using a button to the right of their name.

The Staff Schedular application may be implemented as a combination of server-side PHP scripts and client-side JavaScript, and may be hosted on a web server within the existing hospital network or as a cloud-based application using Docker containers.

The Machine Log Application is a web-based application that tracks radiotherapy treatment equipment status (e.g., photon clinical linear accelerators, proton synchrotrons, etc.). It comprises an issue report form, email and paging routines for communication of urgent machine issues to desired role groups and an analytics page reporting data by machine, keyword, date, and description in graphical and/or tabular format.

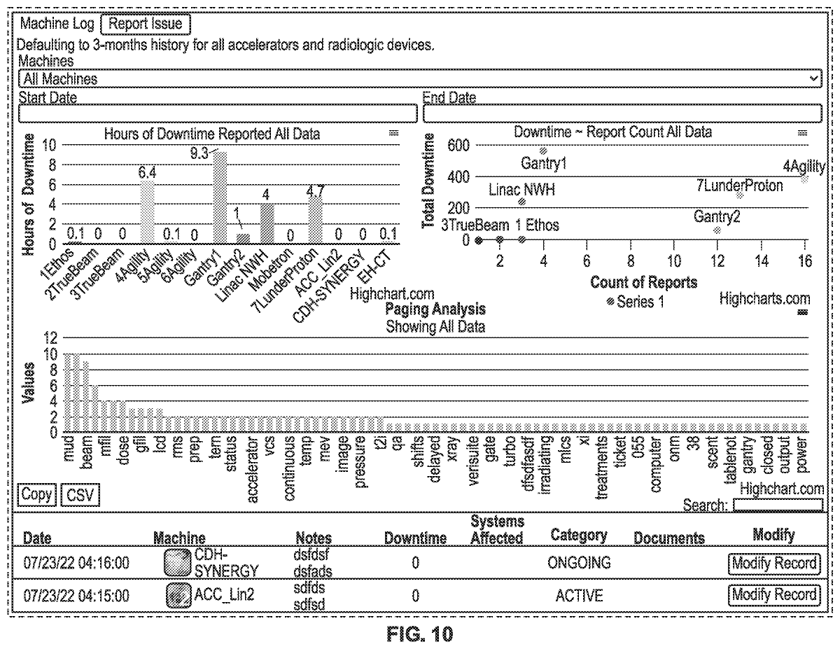

More generally, the Machine Log Application allows clinical users to submit issues and view analytics regarding the status of Radiation Oncology treatment equipment. FIG. 10 illustrates an example of the Machine Log UI. The application consists of an issue submission form and an analytics page presenting filter- and sortable data regarding all reports within a clinic. Specifically data regarding machine “downtime” and keyword frequency is displayed as a function of treatment machine. In addition to graphical data, each report is listed in tabular form. Users may update records to reflect changes in status and record overall “downtime” of the indicated treatment device.

The Machine Log Application may be implemented as a combination of server-side PHP scripts and client-side JavaScript, and may be hosted on a web server within the existing hospital network or as a cloud-based application using Docker containers.

The Morning QA Application is a web-based application used to record radiotherapy treatment machine status performed at the start of every treatment day. It includes a web form for recording machine data and an analytics page for reviewing data in graphical and/or tabular form.

More generally, the Morning QA Application provides a means for radiation oncology therapists to enter data regarding the status of the treatment machines at the start of each day. The morning QA procedure is a recommended activity defined by American Association of Physicists in Medicine (AAPM) Task Group Report 142 (TG-142). This application allows users to record the results of the QA procedure and display historical data for later analysis. The form is customizable based on the type of machine involved (e.g., photon linear accelerator, proton accelerator, imaging modality such as CT, etc.).

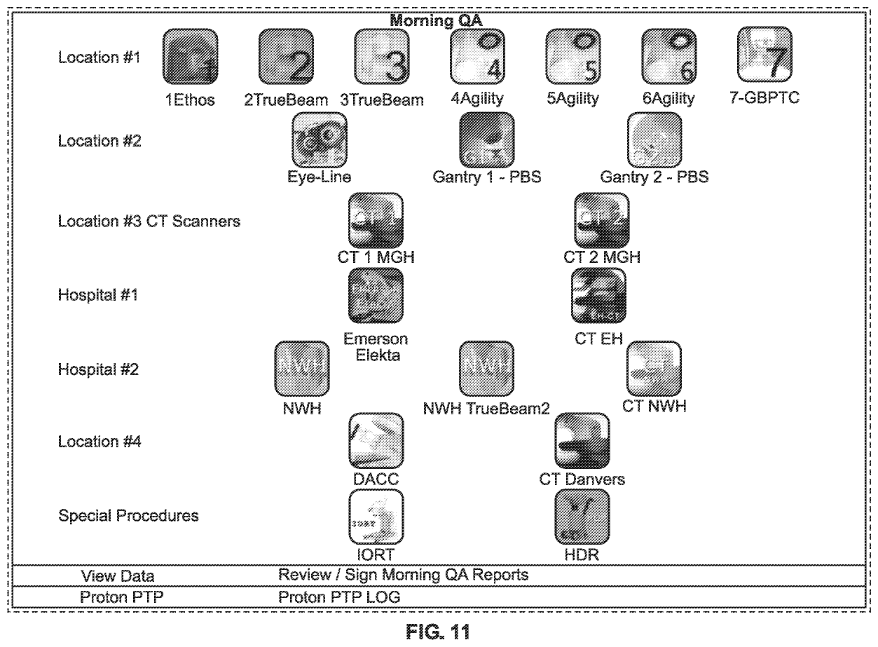

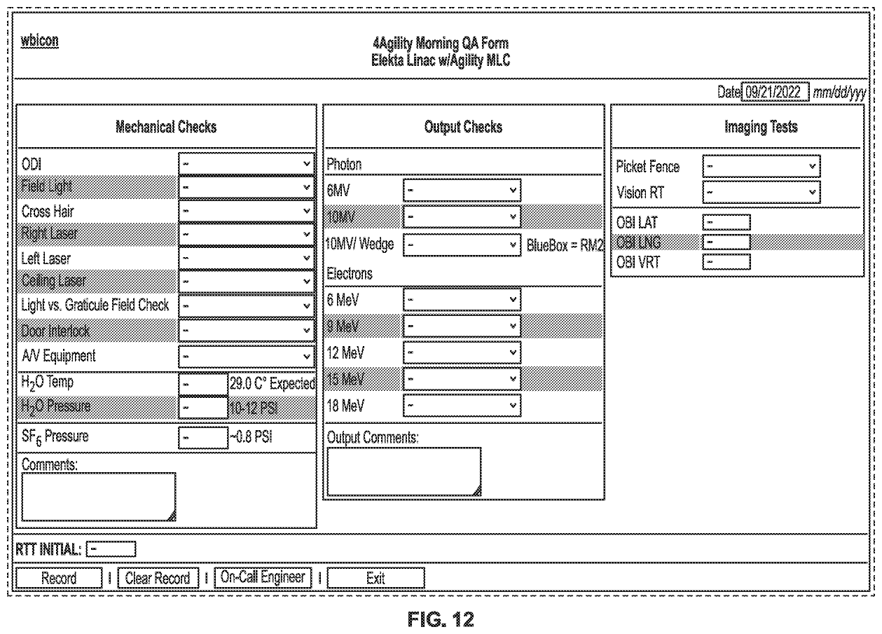

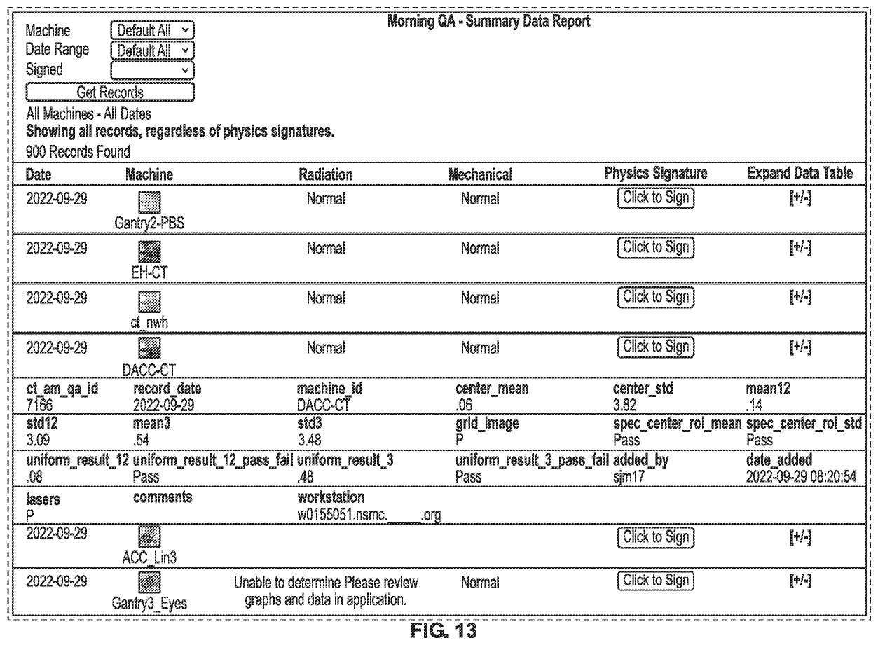

The Morning QA Application comprises a machine selection menu, a form, and a report view. FIG. 11 illustrates one example of the Morning QA device selection screen. FIG. 12 illustrates one example of the Morning QA device status report form, and FIG. 13 illustrates one example of the Morning QA report form. The Morning QA report form includes a list of reports by day with options to filter by various categories such as device or date range. In FIG. 13, example data from one report has been expanded to show detailed results.

The Morning QA Application may be implemented as a combination of server-side PHP scripts and client-side JavaScript, and may be hosted on a web server within the existing hospital network or as a cloud-based application using Docker containers.

The Proton Radiotherapy Management Application (PRMA) is a web-based application that manages treatment execution for proton radiotherapy patients. It comprises a ROWL-defined DICOM Unified Procedure Step (UPS) workflow interface for communication to the treatment machine, a DICOM interface for the exchange of treatment data with a departmental Picture Archiving and Communication System (PACS), and web UI components for user interaction to schedule, execute, and/or review treatments.

More generally, the PRMA schedules, orchestrates, and reviews proton radiotherapy treatments. The PRMA comprises an associated ROCP workflow instance, treatment scheduler, treatment status viewer, and treatment calendar.

The management of proton radiotherapy treatments utilizes a defined ROWL workflow and the workflow engine of the ROCP. Using the PRMA treatment scheduler, a new treatment plan is read in and treatment events are scheduled. Each scheduled treatment event, as described in the ROCP section, references related treatment input objects including a treatment plan (produced by a treatment planning system), a RT structure Set, and a RT Beam Delivery Instruction (dynamically generated by ROCP).

The ROCP workflow engine coordinates the delivery of the proton treatments, preparing data for the treatment machine, recording the status of the treatment and recording the produced treatment records from the treatment machine. The ROCP supports a DICOM UPS interface for communication of treatment event state and delivery data. The ROCP implements an IHE-RO IPDW interface for each treatment event. All interfaces are modeled using ROWL.

The PRMA presents several UI components for user interaction, including: 1) Treatment Scheduler, a UI for scheduling new treatment plans; 2) Daily Treatment List, a UI for RTTs to review treatments scheduled for a selected day: 3) Treatment Statistics, a UI displaying treatment time statistics for all treatment deliveries; 4) QA View, a UI for reviewing data from recorded patient treatments; and 5) Treatment Delivery Calendar.

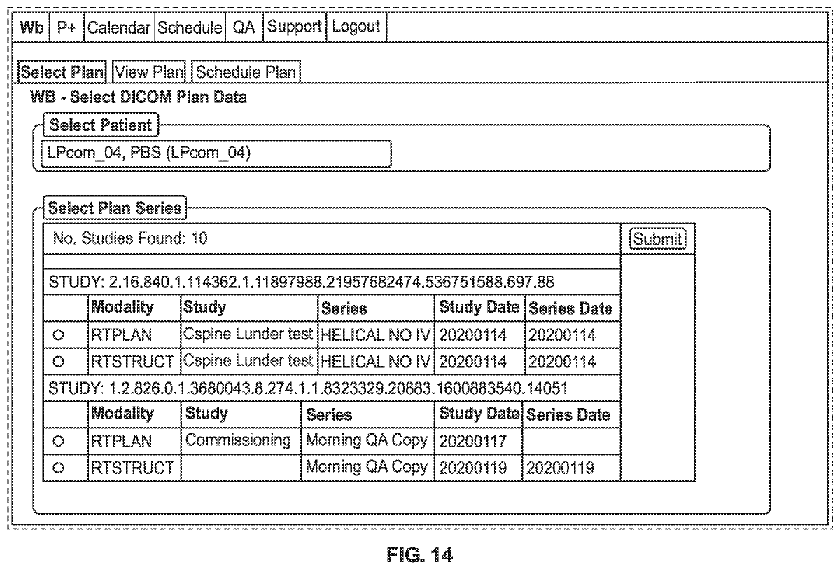

The Treatment Scheduler is illustrated in FIG. 14. It presents a list of available treatment plans by Patient ID that the user may select for scheduling. The application communicates with a local PACS where patient data is stored using DICOM C-FIND interface. Upon selecting a plan for scheduling, the plan metadata may be viewed and, once verified, the plan may be submitted for scheduling. The tool may create ROCP tasks for each prescribed treatment event according to the delivery scheme defined within the plan.

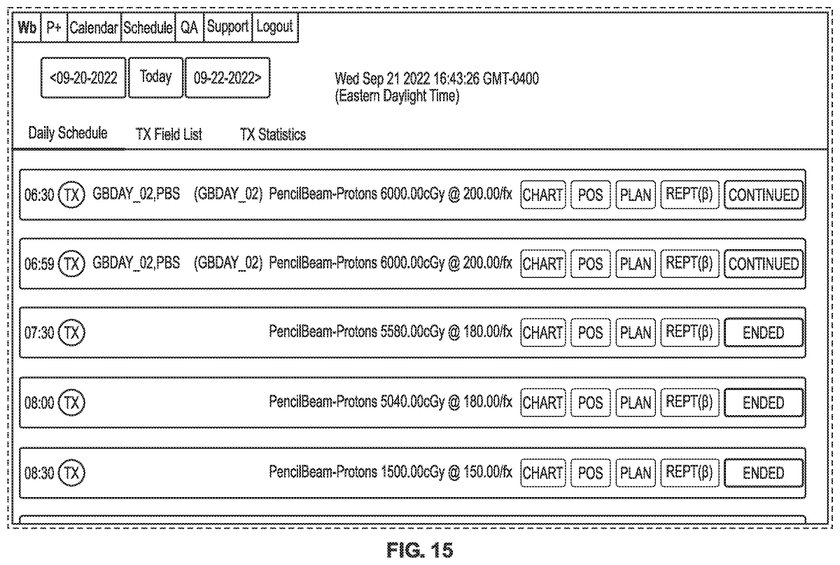

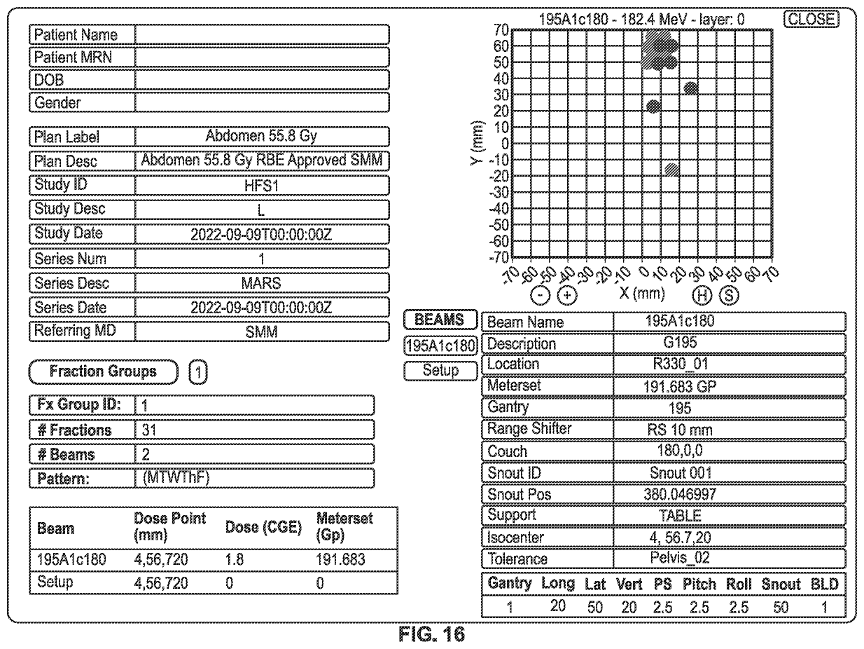

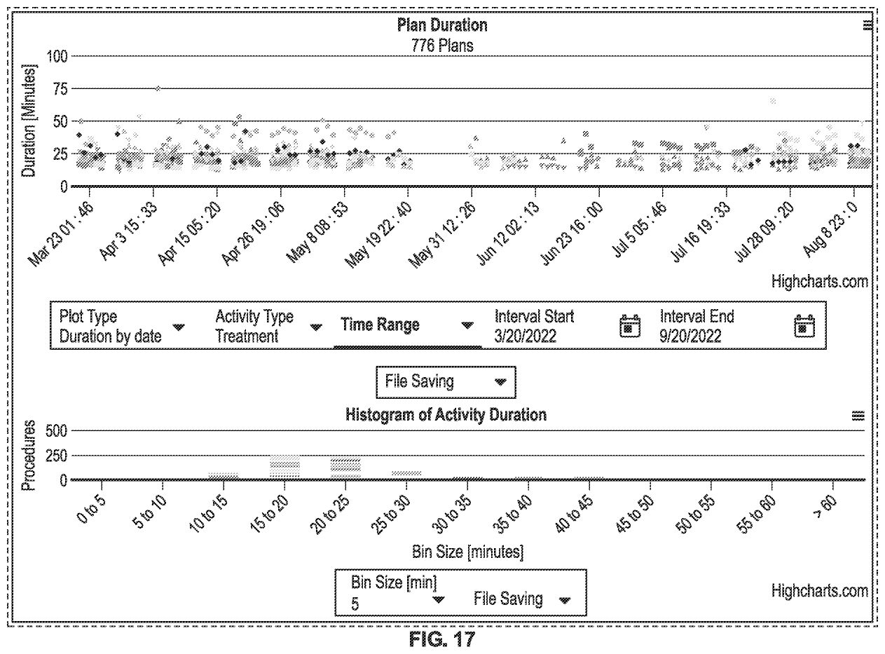

The Daily Treatment List is illustrated in FIG. 15. In in its default view, it may present a list of patient treatment sessions for the selected day. A status of sessions is reported (e.g., IDLE, IN PROGRESS, ENDED, CONTINUED, etc.). Buttons exist to allow viewing of related patient delivery information (e.g., patient chart, patient position data, patient plan data, patient report data, etc.). The Daily Treatment List may also have a treatment plan view, illustrated in FIG. 16, which is a UI triggered from the default view that allows a user to view specific detail regarding a treatment plan. The Treatment Statistics UI is illustrated in FIG. 17. It presents data for all treatments for a selected time period.

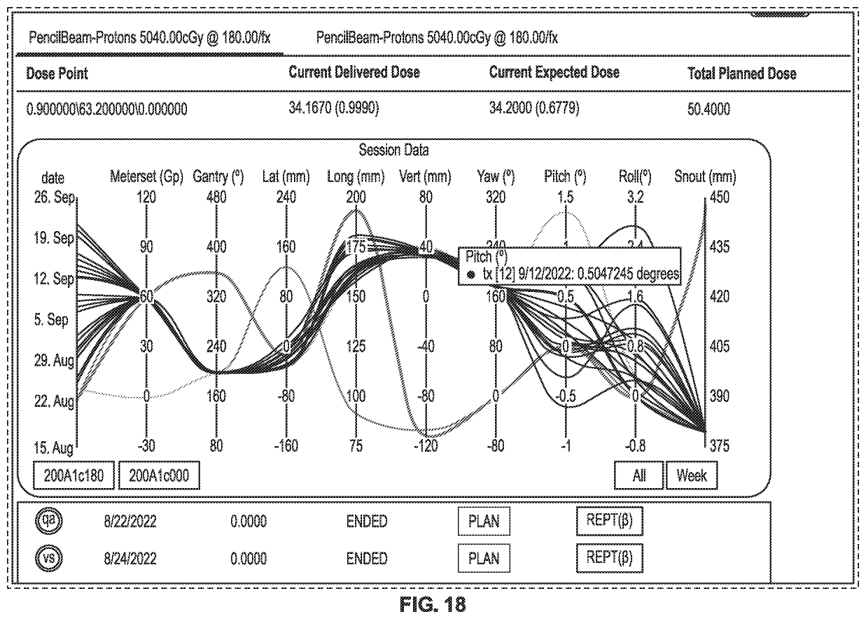

The QA View is illustrated in FIG. 18. It displays a list of treatments for a selected patient treatment phase. Information regarding overall patient delivery is displayed in graphical form. A list of treatments and treatment data may be presented in tabular form.

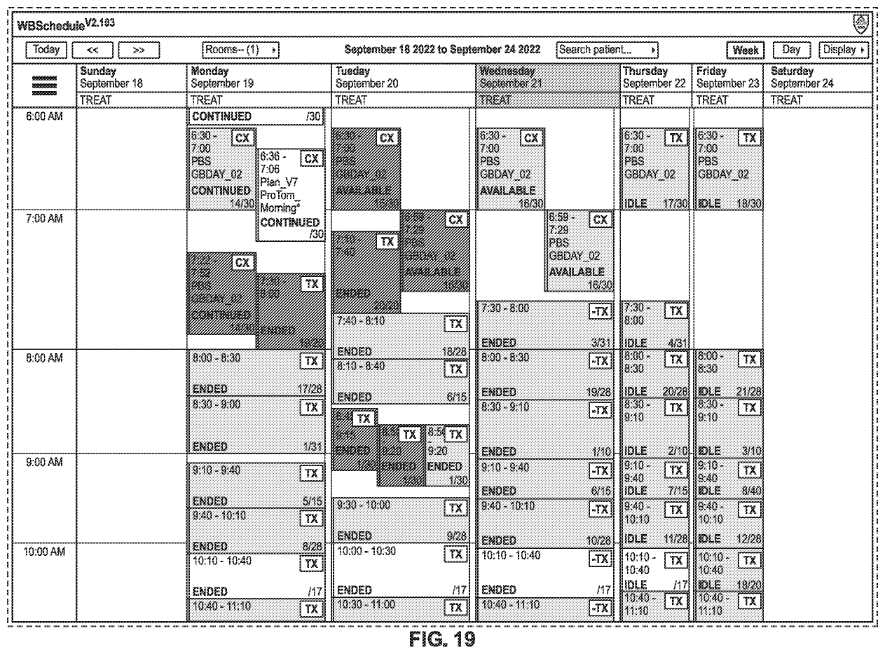

The Treatment Delivery Calendar is illustrated in FIGS. 19-23, and serves as the main view for treatment delivery. Scheduled treatment events are displayed in a desktop calendar UI. The calendar may be filtered to display a particular day, week, month, patient, and/or treatment room. In FIG. 19, a week view is illustrated in which sessions are displayed as blocks within the desktop calendar format. Treatment events may be colored according to resource conflict status. Selection of a treatment event may cause a color change (e.g., to gold to highlight the current selection) and result in the display of the session detail view illustrated in FIG. 20.

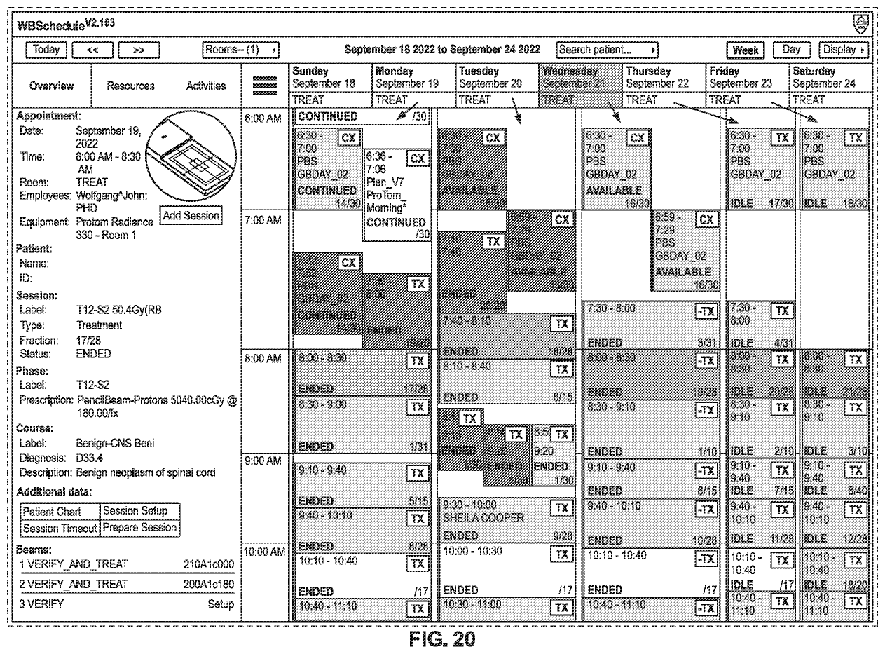

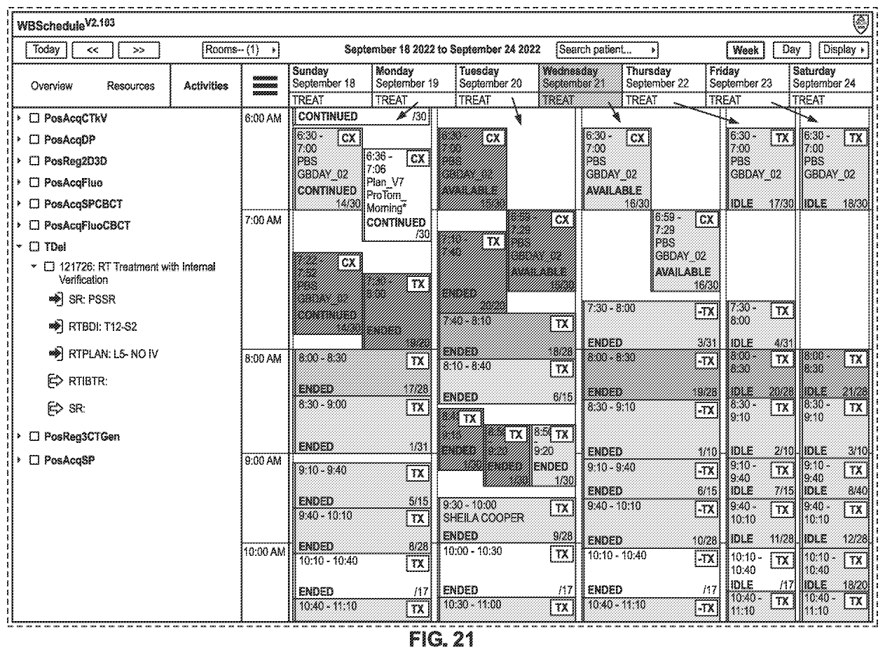

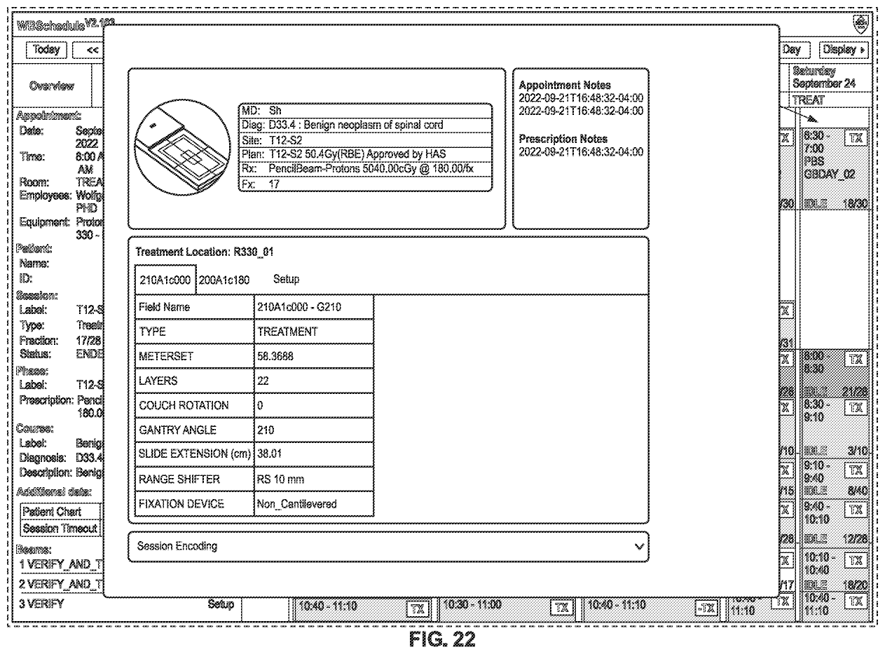

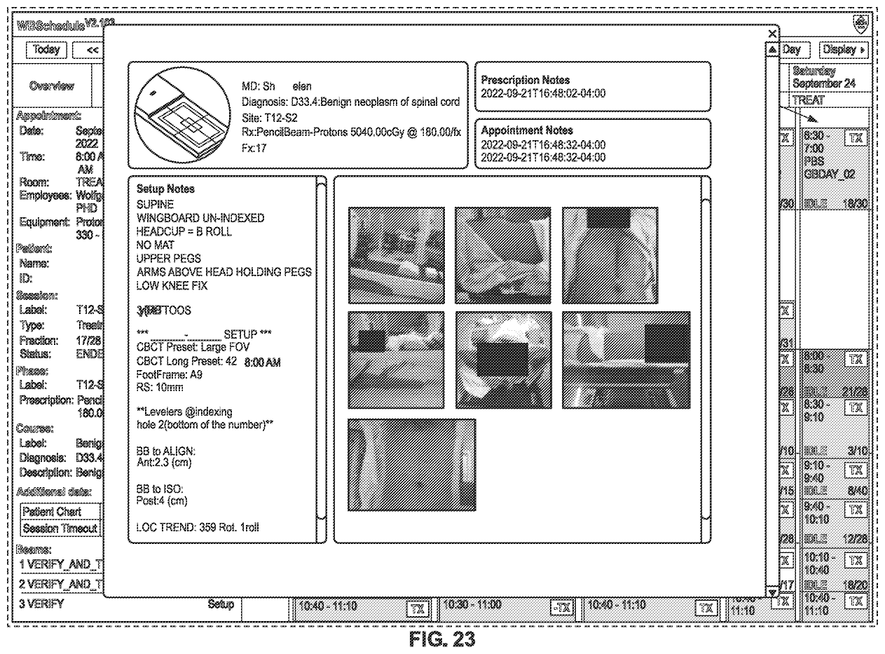

The session detail panel presents selectable views to display sub-tasks for the treatment delivery session selected. Further controls are available to display treatment field delivery details and patient setup details. For example, FIG. 21 illustrates the Treatment Delivery Calendar with the session detail panel open and showing session sub-tasks and related data; FIG. 22 illustrates the Treatment Delivery Calendar with a treatment field detail overlay view open; and FIG. 23 illustrates the Treatment Delivery Calendar with a patient setup view displayed.

The Immobilization Device Tracking Application is a web-based application that tracks the creation and delivery of patient-specific radiotherapy treatment immobilization devices (e.g., masks, vacuum-formed supports, custom devices, etc.). It comprises a device submission form and UI for viewing status and location.

The Waiting Room RSS Feed Application provides a clinical status and information view for patients in the radiation oncology waiting room. It includes informational views describing general descriptions of radiotherapy procedures and equipment as well as patient wait times for clinical treatment appointments.

Thus, the WB (e.g., through the ROCP) may structure and/or aggregate data and/or key performance indicators (KPIs) from a varied constellation of disparate systems, including systems from a number of third party vendors. The aggregated data and/or KPIs may further be used to build predictive models. The predictive models may be or implement artificial intelligence (AI) and/or machine learning (ML) models, which models may be trained using reference data corresponding to known outcomes (e.g., known treatment plans). The reference data may itself correspond to data that has previously been structured and/or aggregated by the WB. In one example, a predictive model may be used to create, in an automated manner, course directives. Generated course directives may be based on a combination of data from the Intake Form and the historic Planning Directives, thereby to extrapolate a new Planning Directive (or part thereof) for a patient.

The WB may operate using any one or more of the above-described UIs so as to interface with the user in a manner that permits the user to interact with one or more other applications which perform simple atomic functions (referred to herein as “atomic applications”) without having to individually access the atomic applications. The atomic applications may be third party applications made by any number of third parties. In many cases, such as data entry, the WB operates in an automated manner to avoid any requirement that the user repeatedly enter the same information for use by different atomic applications. For example, the WB may present one or more visually perceptible elements with which or on which the user may operate. Upon operation by the user, the WB may identify the appropriate atomic application and present information from the atomic application within the same WB environment. Thereby, the WB may provide a UI which merges content associated with the atomic application with the visually perceptible elements of the WB itself, thereby preserving the “look and feel” of the overall WB environment.

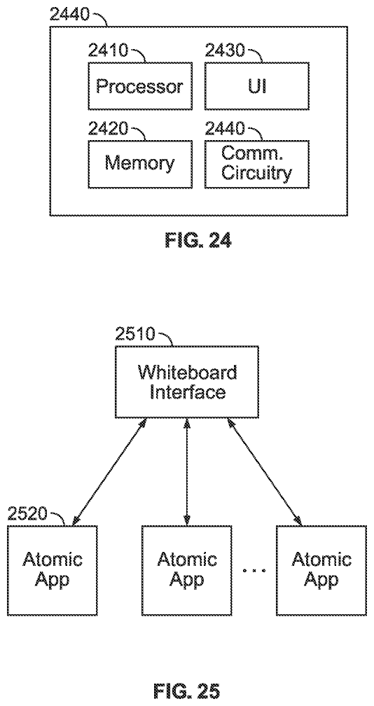

The above applications and functions may be implemented via a unified radiation oncology workflow management system. FIG. 24 illustrates one such example in accordance with the present disclosure. As illustrated in FIG. 24, a radiation oncology workflow management system 2400 includes at least one processor 2410, at least one memory 2420, a user interface (UI) 2430, and communication circuitry 2440.

As used herein, the term “processor” may include one or more individual electronic processors, each of which may include one or more processing cores, and/or one or more programmable hardware elements. The processor 2410 may be or include any type of electronic processing device, including but not limited to central processing units (CPUs), graphics processing units (GPUs), application specific integrated circuits (ASICs), field programmable gate arrays (FPGAs), microcontrollers, digital signal processors, or other devices capable of executing software instructions. One or all of the individual electronic processors may be external to the system 2400 (e.g., to implement cloud or distributed computing). In implementations where the system 2400 has multiple processors 2410 and/or multiple processing cores, individual operations may be performed by any one or more of the microprocessors or processing cores, in series or parallel, in any combination.

The memory 2420 may be any storage medium, including a non-volatile medium, e.g., a magnetic media or hard disk, optical storage, or flash memory; a volatile medium, such as system memory, e.g., random access memory (RAM) such as dynamic RAM (DRAM), synchronous dynamic RAM (SDRAM), static RAM (SRAM), extended data out (EDO) DRAM, extreme data rate dynamic (XDR) RAM, double data rate (DDR) SDRAM, etc.; or an installation medium, such as software media, e.g., a CD-ROM, or floppy disks, on which programs may be stored and/or data communications may be buffered. The term “memory” may also include other types of memory or combinations thereof. For the avoidance of doubt, cloud storage is contemplated in the definition of memory.

The memory 2420 is an example of a non-transitory computer-readable medium which stores instructions that are executable by the processor 2410. Execution of the instructions by the processor 2420 may be configured to cause the system 2400 or another system operating under control of the system 2400 to perform one or more operations, including but not limited to processing, communication, automation, and display operations set forth herein. In one particular example, execution of the instructions by the processor 2420 may be configured to cause the system 2400 to perform operations comprising displaying a window containing a user interface (UI) environment on a computer screen: parsing an input received from a user via the UI environment, thereby to determine which of a plurality of atomic application functions is indicated by the input; automatically interacting with an atomic application (or applications) associated with the atomic application function determined to be indicated by the input; and automatically displaying information received from the atomic application via the UI environment.

The UI environment may include any one or more of the UIs described above, including the Workflow Engine (ROCP), the Workflow UI (CorTx), the Workflow Analytics Application, the Intake Form, the Planning Directive, the Staff Scheduler, the Machine Log Application, the Morning QA Application, the Proton Radiotherapy Application, the Immobilization Device Tracking Application, and/or the Waiting Room RSS Feed Application. The atomic application may be any one or more of an electronic medical record application, a treatment planner application, a calendar application, an equipment tracker application, a QA application, a treatment management application, a device submission or tracking form, and/or an RSS feed application.

The UI 2430 may be any interface by which a user may interact with the system 2400 and vice versa. The UI 2430 may include graphical components (e.g., a GUI), physical buttons, soft buttons, peripheral devices (e.g., mice, keyboards, etc.), audio devices, haptic feedback devices, touchscreen devices, display devices, or combinations thereof. In some implementations, the peripheral, audio, haptic feedback, touchscreen, and/or display devices may be external to the system 2400, in which case the UI 2430 may include hardware, software, and/or firmware to permit interfacing and communication with the external devices (e.g., an I/O port), either alone or in combination with the communication circuitry 2440. The graphical components of the UI 2430 may be or include any one or more of the UI components described above with regard to FIGS. 2-23. The UI 2430 may permit the user to interact therewith (e.g., by clicking or touching a portion of the UI 2430) thereby to cause the system 2400 to perform certain operations in response to the interactions.

The communication circuitry 2440 may be any wired or wireless communication interface to permit communication between the system 2400 and extemal systems and devices. The communication circuitry 2440 may be configured to permit wired communication via, for example, a copper wire, a fiber optic cable, and the like. The communication circuitry 2440 may be additionally or alternatively configured to permit wireless communication via, for example, a Wi-Fi protocol, a Bluetooth protocol, a Near Field Communication (NFC) protocol, a Third Generation Partnership Project (3GPP) protocol such as Long Term Evolution (LTE), 5G New Radio (NR), and the like, including extensions and updated versions of any of the above protocols. In some examples, the communication circuitry 2440 may operate under the control of the processor 2410 to upload data to extemal devices (e.g., a cloud-based server), to request processing from extemal devices, to receive response data from external devices, and so on.

The WB may provide a means to interface between a user and one or more atomic applications, each performing an individualized function. FIG. 25 illustrates one example of such a scheme, in which a whiteboard interface 2510 is communicatively coupled to a plurality of atomic applications 2520. While three atomic applications 2520 are illustrated in FIG. 25, in practice any number of atomic applications 2520 may be present, including one, two, and more than three.

The whiteboard interface 2510 may be presented via a radiation oncology workflow management system, such as the system 2400 illustrated in FIG. 24. Thus, the whiteboard interface 2510 may be embodied in the form of one or more graphical components presented on the UI 2430. The whiteboard interface 2510 receives commands and other input from a user and interacts with the atomic applications 2520 in such a manner that the user need not interact with the atomic applications 2520. For example, the whiteboard interface 2510 may (e.g., under the control of a processor contained therein and/or under the control of another processor) display a window containing a user interface (UI) environment on a computer screen; parse an input received from a user via the UI environment, thereby to determine which of a plurality of atomic application functions is indicated by the input; automatically interacting with the appropriate atomic application 2520 that is associated with the atomic application function determined to be indicated by the input; and automatically displaying information received from the atomic application via the UT environment.

The whiteboard interface 2510 presents an environment that may include any one or more of the UIs described above, including the Workflow Engine (ROCP), the Workflow UI (CorTx), the Workflow Analytics Application, the Intake Form, the Planning Directive, the Staff Scheduler, the Machine Log Application, the Morning QA Application, the Proton Radiotherapy Application, the Immobilization Device Tracking Application, and/or the Waiting Room RSS Feed Application. The atomic application may be any one or more of an electronic medical record application, a treatment planner application, a calendar application, an equipment tracker application, a QA application, a treatment management application, a device submission or tracking form, and/or an RSS feed application.

In general, the present disclosure may be implemented in any one or more of the following example configurations.

Configuration 1. A radiation oncology workflow management system comprising: an interactive graphical interface configured to affect a workflow operation and workstation behavior for a radiation oncology patient-specific workflow; and a particularly programmed processor including computer program instructions, the instructions, when executed by the processor, cause the system to perform: displaying a window containing a user interface (UI) environment on a computer screen, parsing an input received from a user via the UI environment, thereby to determine which of a plurality of atomic application functions is indicated by the input, automatically interacting with an atomic application associated with the atomic application function determined to be indicated by the input, and automatically displaying information received from the atomic application via the UI environment.

Configuration 2. The system of configuration 1, wherein the UI environment includes a view selected from a racetrack view including a plurality of individual tasks represented as state-specifying interactive pill icons, and a single patient chart view organized according to a Digital Imaging and Communications in Medicine (DICOM) schema.

Configuration 3. The system of configuration 1 or 2, wherein the UI environment includes an intake form including a first field on which the user may add or edit a patient information and/or a treatment information, and the atomic application is an electronic medical record application.

Configuration 4. The system of configuration 3, wherein the intake form includes a second field on which the user may enter a patient treatment planning simulation.

Configuration 5. The system of any one of configurations 1 to 4, wherein the UI environment includes planning directive form by which the user may construct or edit a radiotherapy plan, and the atomic application is a treatment planner application.

Configuration 6. The system of any one of configurations 1 to 5, wherein the UI environment includes a staff scheduler UI configured to indicate staff information, and the atomic application is a calendar application.

Configuration 7. The system of any one of configurations 1 to 6, wherein the UI environment includes a machine log UI, and the atomic application is an equipment tracker application.

Configuration 8. The system of any one of configurations 1 to 7, wherein the UI environment includes a morning quality assurance interface by which the user may add or edit a treatment status and/or a treatment machine status, and the atomic application is quality assurance application.

Configuration 9. The system of any one of configurations 1 to 8, wherein the UI environment includes a proton radiation management interface by which the user may manage a treatment execution, and the atomic application is a treatment management application.

Configuration 10. The system of configuration 9, wherein the proton radiation management interface includes at least one of a treatment scheduler, a daily treatment list, a treatment statistic interface, a quality assurance view, or a treatment delivery calendar.

Configuration 11. The system of any one of configurations 1 to 10, wherein the UI environment includes a device tracking interface by which the user may track a creation and/or delivery of a patient-specific immobilization device, and the atomic application includes a device submission form.

Configuration 12. The system of any one of configurations 1 to 11, wherein the UI environment includes a waiting room interface, and the atomic application is an RSS feed application.

Configuration 13. A non-transitory computer-readable medium storing instructions that, when executed by a processor of a computing machine, cause the computing machine to be particularly programmed to perform operations comprising: displaying a window containing a user interface (UI) environment on a computer screen: parsing an input received from a user via the UI environment, thereby to determine which of a plurality of atomic application functions is indicated by the input; automatically interacting with an atomic application associated with the atomic application function determined to be indicated by the input; and automatically displaying information received from the atomic application via the UI environment.

Configuration 14. The non-transitory computer-readable medium of configuration 13, wherein the UI environment includes a view selected from a racetrack view including a plurality of individual tasks represented as state-specifying interactive pill icons, and a single patient chart view organized according to a Digital Imaging and Communications in Medicine (DICOM) schema.

Configuration 15. The non-transitory computer-readable medium of configuration 13 or 14, wherein the UI environment includes an intake form including a first field on which the user may add or edit a patient information and/or a treatment information, and the atomic application is an electronic medical record application.

Configuration 16. The non-transitory computer-readable medium of configuration 15, wherein the intake form includes a second field on which the user may enter a patient treatment planning simulation.

Configuration 17. The non-transitory computer-readable medium of any one of configurations 13 to 16, wherein the UI environment includes a planning directive form by which the user may construct or edit a radiotherapy plan, and the atomic application is a treatment planner application.

Configuration 18. The non-transitory computer-readable medium of any one of configurations 13 to 17, wherein the UI environment includes a staff scheduler UI configured to indicate staff information, and the atomic application is a calendar application.

Configuration 19. The non-transitory computer-readable medium of any one of configurations 13 to 18, wherein the UI environment includes a machine log UI, and the atomic application is an equipment tracker application.

Configuration 20. The non-transitory computer-readable medium of any one of configurations 13 to 19, wherein the UI environment includes a morning quality assurance interface by which the user may add or edit a treatment status and/or a treatment machine status, and the atomic application is quality assurance application.

Configuration 21. The non-transitory computer-readable medium of any one of configurations 13 to 20, wherein the UI environment includes a proton radiation management interface by which the user may manage a treatment execution, and the atomic application is a treatment management application.

Configuration 22. The non-transitory computer-readable medium of configuration 21, wherein the proton radiation management interface includes at least one of a treatment scheduler, a daily treatment list, a treatment statistic interface, a quality assurance view, or a treatment delivery calendar.

Configuration 23. The non-transitory computer-readable medium of any one of configurations 13 to 22, wherein the UI environment includes a device tracking interface by which the user may track a creation and/or delivery of a patient-specific immobilization device, and the atomic application includes a device submission form.

Configuration 24. The non-transitory computer-readable medium of any one of configurations 13 to 23, wherein the UI environment includes a waiting room interface, and the atomic application is an RSS feed application.

Other examples and uses of the disclosed technology will be apparent to those having ordinary skill in the art upon consideration of the specification and practice of the invention disclosed herein. The specification and examples given should be considered exemplary only, and it is contemplated that the appended claims will cover any other such embodiments or modifications as fall within the true scope of the invention.

The Abstract accompanying this specification is provided to enable the United States Patent and Trademark Office and the public generally to determine quickly from a cursory inspection the nature and gist of the technical disclosure and in no way intended for defining, determining, or limiting the present invention or any of its embodiments.

What is claimed is:

1. A radiation oncology workflow management system comprising:
an interactive graphical interface configured to affect a workflow operation and workstation behavior for a radiation oncology patient-specific workflow; and
a particularly programmed processor including computer program instructions, the instructions, when executed by the processor, cause the system to perform:
displaying a window containing a user interface (UI) environment on a computer screen,
parsing an input received from a user via the UI environment, thereby to determine which of a plurality of atomic application functions is indicated by the input,
automatically interacting with an atomic application associated with the atomic application function determined to be indicated by the input, and
automatically displaying information received from the atomic application via the UI environment.

2. The system of claim 1, wherein the UI environment includes a view selected from a racetrack view including a plurality of individual tasks represented as state-specifying interactive pill icons, and a single patient chart view organized according to a Digital Imaging and Communications in Medicine (DICOM) schema.

3. The system of claim 1 or 2, wherein the UI environment includes an intake form including a first field on which the user may add or edit a patient information and/or a treatment information, and the atomic application is an electronic medical record application.

4. The system of claim 3, wherein the intake form includes a second field on which the user may enter a patient treatment planning simulation.

5. The system of any one of claims 1 to 4, wherein the UI environment includes planning directive form by which the user may construct or edit a radiotherapy plan, and the atomic application is a treatment planner application.

6. The system of any one of claims 1 to 5, wherein the UI environment includes a staff scheduler UI configured to indicate staff information, and the atomic application is a calendar application.

7. The system of any one of claims 1 to 6, wherein the UI environment includes a machine log UI, and the atomic application is an equipment tracker application.

8. The system of any one of claims 1 to 7, wherein the UI environment includes a morning quality assurance interface by which the user may add or edit a treatment status and/or a treatment machine status, and the atomic application is quality assurance application.

9. The system of any one of claims 1 to 8, wherein the UI environment includes a proton radiation management interface by which the user may manage a treatment execution, and the atomic application is a treatment management application.

10. The system of claim 9, wherein the proton radiation management interface includes at least one of a treatment scheduler, a daily treatment list, a treatment statistic interface, a quality assurance view, or a treatment delivery calendar.

11. The system of any one of claims 1 to 10, wherein the UI environment includes a device tracking interface by which the user may track a creation and/or delivery of a patient-specific immobilization device, and the atomic application includes a device submission form.

12. The system of any one of claims 1 to 11, wherein the UI environment includes a waiting room interface, and the atomic application is an RSS feed application.

13. A non-transitory computer-readable medium storing instructions that, when executed by a processor of a computing machine, cause the computing machine to be particularly programmed to perform operations comprising:
displaying a window containing a user interface (UI) environment on a computer screen;
parsing an input received from a user via the UI environment, thereby to determine which of a plurality of atomic application functions is indicated by the input;
automatically interacting with an atomic application associated with the atomic application function determined to be indicated by the input; and
automatically displaying information received from the atomic application via the UI environment.

14. The non-transitory computer-readable medium of claim 13, wherein the UI environment includes a view selected from a racetrack view including a plurality of individual tasks represented as state-specifying interactive pill icons, and a single patient chart view organized according to a Digital Imaging and Communications in Medicine (DICOM) schema.

15. The non-transitory computer-readable medium of claim 13 or 14, wherein the UI environment includes an intake form including a first field on which the user may add or edit a patient information and/or a treatment information, and the atomic application is an electronic medical record application.

16. The non-transitory computer-readable medium of claim 15, wherein the intake form includes a second field on which the user may enter a patient treatment planning simulation.

17. The non-transitory computer-readable medium of any one of claims 13 to 16, wherein the UI environment includes a planning directive form by which the user may construct or edit a radiotherapy plan, and the atomic application is a treatment planner application.

18. The non-transitory computer-readable medium of any one of claims 13 to 17, wherein the UI environment includes a staff scheduler UI configured to indicate staff information, and the atomic application is a calendar application.

19. The non-transitory computer-readable medium of any one of claims 13 to 18, wherein the UI environment includes a machine log UI, and the atomic application is an equipment tracker application.

20. The non-transitory computer-readable medium of any one of claims 13 to 19, wherein the UI environment includes a morning quality assurance interface by which the user may add or edit a treatment status and/or a treatment machine status, and the atomic application is quality assurance application.

21. The non-transitory computer-readable medium of any one of claims 13 to 20, wherein the UI environment includes a proton radiation management interface by which the user may manage a treatment execution, and the atomic application is a treatment management application.

22. The non-transitory computer-readable medium of claim 21, wherein the proton radiation management interface includes at least one of a treatment scheduler, a daily treatment list, a treatment statistic interface, a quality assurance view, or a treatment delivery calendar.

23. The non-transitory computer-readable medium of any one of claims 13 to 22, wherein the UI environment includes a device tracking interface by which the user may track a creation and/or delivery of a patient-specific immobilization device, and the atomic application includes a device submission form.

24. The non-transitory computer-readable medium of any one of claims 13 to 23, wherein the UI environment includes a waiting room interface, and the atomic application is an RSS feed application.
